(19) United States
(12) Patent Application Publication
(10) Pub. No.: US 2020/0163626 A1
(43) Pub. Date: May 28, 2020

(54) GLUCOSE LEVEL PREDICTION AND MANAGEMENT

(71) Applicant: INTERNATIONAL BUSINESS MACHINES CORPORATION, ARMONK, NY (US)

(72) Inventors: Priscilla M. Guzman Angulo, Santo Domingo (CR); Kenneth Alberto Gonzalez Salas, San Pablo (CR); Roxana Monge Nunez, Perez Zeledon (CR)

(21) Appl. No.: 16/201,049

(22) Filed: Nov. 27, 2018

(51) Int. Cl. A61B 5/00; A61B 5/145; A61B 5/11

(52) U.S. Cl. CPC A61B 5/7275; A61B 5/14532; A61B 5/1118; A61B 5/4866; A61B 5/486; A61B 5/681; A61B 5/746; A61B 5/7267; A61B 5/747; A61B 5/1112; A61B 5/742

(57) ABSTRACT

An embodiment of the invention may include a method, computer program product and computer system for glucose management. The method, computer program product and computer system may include a computing device which may collect application data associated with a user and receive data from the user. The computing device may create a user profile from the received data and analyze the collected application data and the user profile to identify one or more user activities and associated blood glucose levels for the user for the one or more activities. The computing device may analyze the application data to determine a current status of the user or a future planned activity of the user is associated with an abnormal blood glucose level. The computing device may generate a recommendation for maintaining a normal blood glucose level for display to the user.

### BACKGROUND

The present invention relates generally to a method, system, and computer program for a glucose management. More particularly, the present invention relates to a method, system, and computer program for identifying a glucose level pattern for a user and providing recommendations based on the user's glucose level pattern.

Glucose (C6H12O6) is a type of sugar which the human body uses for energy. Glucose is typically ingested and circulates in the human body as blood sugar. The human body must maintain an optimal level of blood glucose in order to function properly. Low blood sugar can result in hypoglycemia and high blood sugar can result in hyperglycemia, both of which are dangerous conditions. Chronically high or low blood sugar level can result in Diabetes and requires the individual to constantly monitor his/her blood sugar levels. Further, obesity is related to an uncontrolled consumption of products with a high sugar content and thus a resulting high blood sugar level. The optimal blood sugar level is measured as a blood sugar coefficient, which is generated using various indicators and body states and translated as the sugar level assimilation of the body in certain period of time. The normal range of the blood sugar coefficient is between 3.9 and 5.5 mmol/L. It is important to understand that those levels can increase or decrease during the day but on average should remain relatively constant (most likely 5.5 mmol/L). When a person exceeds the recommended blood sugar level during their normal routine and around meal times (5.0 to 7.2 mmol/L, based on the American Diabetes Association), the person may be diagnosed as having diabetes. For most people with diabetes, a healthy blood sugar range is between 90 and 130 mg/dl before meals and less than 180 mg/dl at one to two hours after a meal. People with chronic diabetes should avoid ingesting excess sugar in order to control their blood sugar levels.

### BRIEF SUMMARY

An embodiment of the invention may include a method, computer program product and computer system for glucose management. The method, computer program product and computer system may include a computing device which may collect application data associated with a user and receive data from the user. The computing device may create a user profile from the received data and analyze the collected application data and the user profile to identify one or more user activities and associated blood glucose levels for the user for the one or more activities. The computing device may analyze the application data to determine a current status of the user and the user's current status is associated with an abnormal blood glucose level. The computing device may generate a recommendation for maintaining a normal blood glucose level for display to the user. The computing device may analyze the application data to determine a future planned activity of the user and predict a user blood glucose level associated with the future planned activity. The computing device may determine the future planned activity is associated with an abnormal blood glucose level and generate a recommendation for maintaining a normal blood glucose level for display to the user.

### BRIEF DESCRIPTION OF THE DRAWINGS

FIG. 1a illustrates a system for a glucose management, in accordance with an embodiment of the invention.

FIG. 1b illustrates example operating modules of the glucose management program of FIG. 1a;

FIG. 2 is a flowchart illustrating an example method of executed by the glucose management system in accordance with an embodiment of the invention.

FIG. 3 is a flowchart illustrating an example method of executed by the glucose management system in accordance with an embodiment of the invention.

FIG. 4 is a block diagram depicting the hardware components of the glucose management system of FIG. 1, in accordance with an embodiment of the invention.

FIG. 5 illustrates a cloud computing environment, in accordance with an embodiment of the invention.

FIG. 6 illustrates a set of functional abstraction layers provided by the cloud computing environment of FIG. 5, in accordance with an embodiment of the invention.

### DETAILED DESCRIPTION

Embodiments of the present invention will now be described in detail with reference to the accompanying Figures.

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

Embodiments of the present invention provide a method, computer program, and computer system for glucose management. Further, embodiments of the present invention provide a method, computer program, and computer system for analyzing a user's past activities and associated blood glucose levels with those past activities in order to predict a user's blood glucose level for a future planned activity. Embodiments of the present invention also generate recommendations to user's on how to control and maintain a healthy blood glucose level based on various data inputs. Current technology can detect a user's blood glucose level, but it is up to the user to interpret that information and determine a course of action, such as injecting insulin, drinking, or eating, etc. Current technology does not allow for the analysis of a user's past activities and associated blood glucose levels and using that analysis to predict the user's future blood glucose level and generate a recommendation for the user to follow to manage their blood glucose level. Embodiments of the present invention provide a differentiated solution by analyzing the user's body metabolism and the user's individual ability to process glucose and enables the quantification of an individual's daily life nutrients consumption for preventing diseases or simply improving the quality of the user's health in an intuitive and easy way, without requiring the use of complex techniques. In addition, embodiments of the present invention provide the ability to correlate an individual's ability to process processing with other measurements, such as physical activity and food and/or beverage consumption using machine learning. Thus, embodiments of the present invention improve existing machine learning systems by enabling the machine learning systems to analyze a user's activities and associated blood glucose level and managing future changes to the user's blood glucose levels. Despite the technological developments today, current technology does not provide a user-friendly way for a user to understand how his/her body reacts to glucose consumption. Further, current technology does not provide users with easy to understand recommendations on maintaining a healthy blood glucose level.

Embodiments of the present invention may improve machine learning systems by creating and adding the corpus of data used by the machine learning system. An example of a machine learning system may be the IBM Watson™ system available from International Business Machines Corporation of Armonk, N.Y., which is augmented with the mechanisms of the illustrative embodiments of the present invention described hereafter. The machine learning system may receive data from a variety of user devices. The machine learning system then performs deep analysis on the data using a variety of algorithms. There may be hundreds or even thousands of algorithms applied, each of which performs different analysis, e.g., comparisons, statistical analysis, optimization, language analysis, etc. For example, some algorithms may look at the matching of a user's blood glucose level with a certain activity such as, but not limited to, exercise, eating, and drinking, etc. Other algorithms may look at future planned activities of the user and utilize the user's history to predict how the future activity will impact the user's blood glucose level. Further, the algorithms may also recommend activities to a user based on predicted blood glucose level changes, for example, recommending a user avoid certain foods, or exercise before leaving for a certain location, etc. The data collected, analyzed, and generated by embodiments of the present invention, as described herein, may be added to the corpus of data of a machine learning system. Thus, the collected, analyzed, and generated by embodiments of the present invention may be utilized by the machine learning system to generate recommendations to a user to help the user maintain a healthy blood glucose level.

Reference will now be made in detail to the embodiments of the present invention, examples of which are illustrated in the accompanying drawings, wherein like reference numerals refer to like elements throughout. Embodiments of the invention are generally directed to a system for predicting the motivational predisposition of an individual.

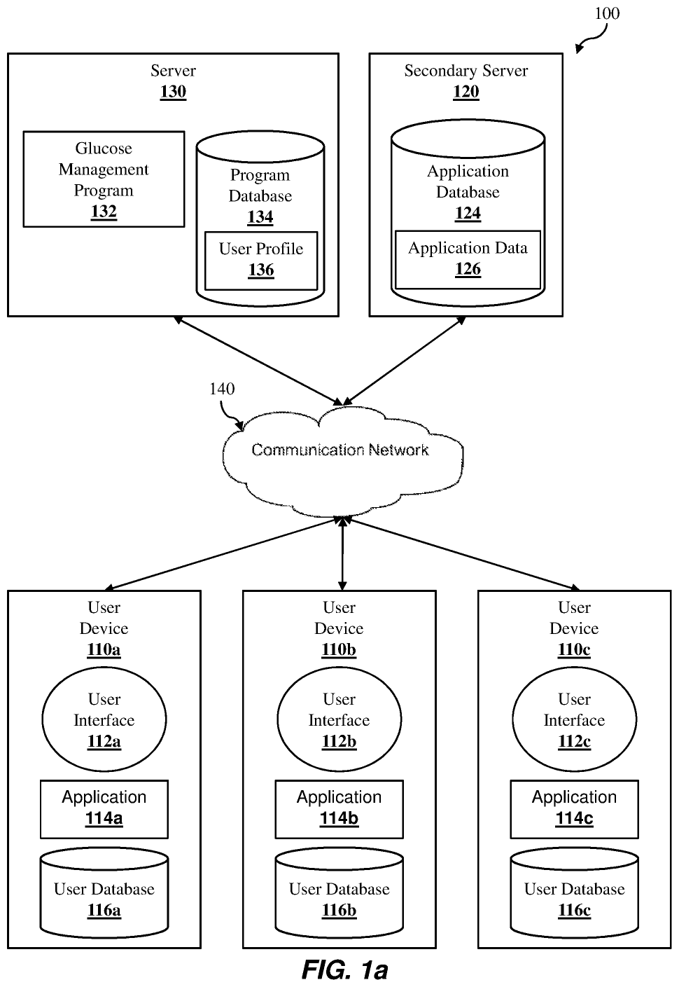

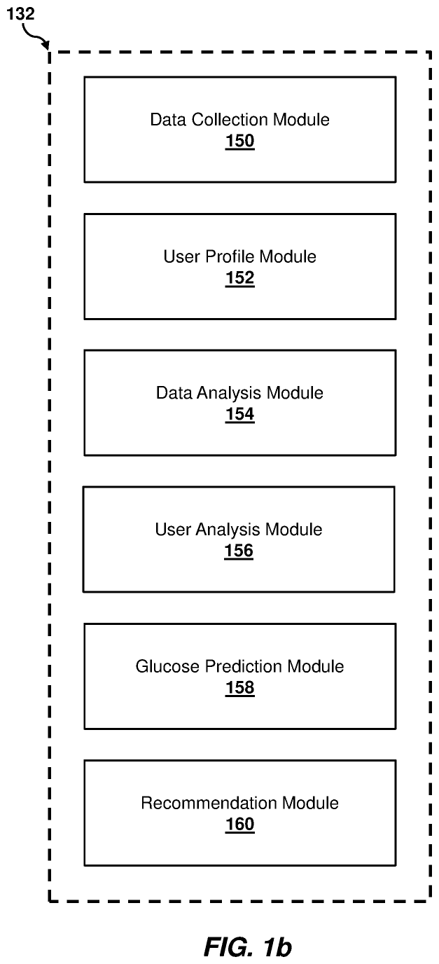

FIG. 1 illustrates a glucose management system 100, in accordance with an embodiment of the invention. In an example embodiment, the glucose management system 100 includes user devices 110a, 110b, 110c, a secondary server 120, and a server 130 interconnected via network 140.

In the example embodiment, the network 140 is the Internet, representing a worldwide collection of networks and gateways to support communications between devices connected to the Internet. The network 140 may include, for example, wired, wireless or fiber optic connections. In other embodiments, the network 140 may be implemented as an intranet, a local area network (LAN), or a wide area network (WAN). In general, the network 140 can be any combination of connections and protocols that will support communications between the user devices 110a, 110b, 110c, the secondary server 120, and the server 130.

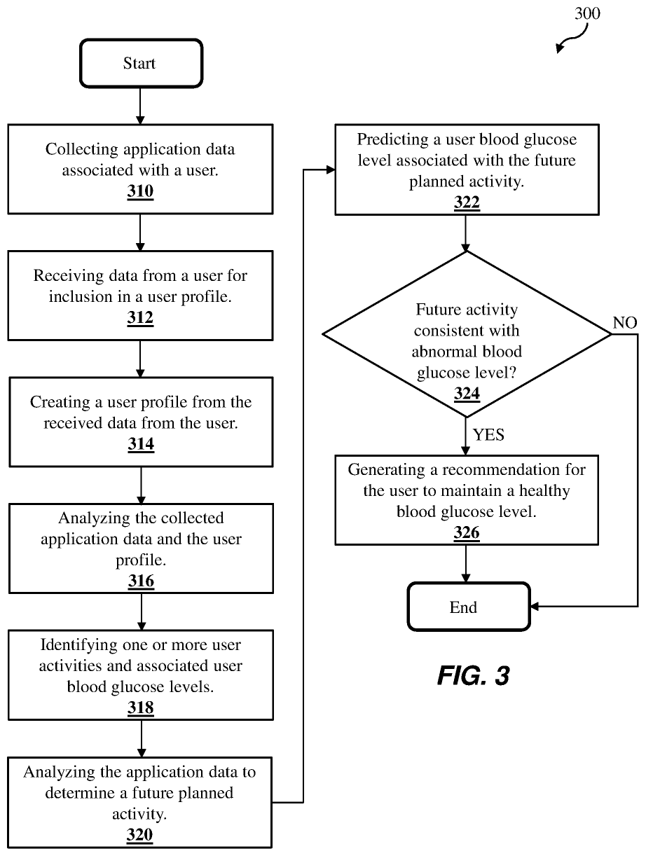

The user devices 110a, 110b, 110c may include a user interfaces 112a, 112b, 112c, applications 114a, 114b, 114c, and user databases 116a, 116b, 116c. The user devices 110a, 110b, 110c may be a desktop computer, a notebook, a laptop computer, a tablet computer, a thin client, or any other electronic device or computing system capable of storing compiling and organizing audio, visual, or textual content and receiving and sending that content to and from other computing devices, such as the secondary server 120, and the server 130 via the network 140. In an example embodiment, the user devices 110a, 110b, 110c may be a health monitoring device such as, but are not limited to, a glucometer, a blood sugar monitor, a smartwatch, a smartphone, a pedometer, a water tracking device, a global positioning system (GPS), a calorie counter, beverage sugar monitor, weight monitor, etc. In some embodiments, the user devices 110a, 110b, 110c include a collection of devices or data sources. For example, a user may have three user devices 110a, 110b, 110c with the user device 110a being a blood sugar glucometer, the user device 110b being a smartwatch, and the user device 110c being a smartphone. Further, the user device 110a, 110b, 110c may be a device of a secondary user, such as, but not limited to, an emergency contact of the user, or someone who the user wished to share their data with. While three user devices 110a, 110b, 110c are depicted, it can be appreciated that any number of user devices may be part of the glucose management system 100 including more than or less than three. The user devices 110a, 110b, 110c are described in more detail with reference to FIG. 3.

The user interfaces 112a, 112b, 112c include components used to receive input from a user on the user devices 110a, 110b, 110c and transmit the input to the glucose management program 132 residing on the server 130, or conversely to receive information from the glucose management program 132 and display the information to users on the user devices 110a, 110b, 110c. In an example embodiment, the user interfaces 112a, 112b, 112c use a combination of technologies and devices, such as device drivers, to provide a platform to enable users of the user devices 110a, 110b, 110c to interact with the meeting concierge program 122. In the example embodiment, the user interfaces 112a, 112b, 112c receive input, such as but not limited to, textual, visual, or audio input received from a physical input device, such as but not limited to, a keypad, mouse, camera, and/or a microphone.

The applications 114a, 114b, 114c may be any electronic application including, but not limited to, downloadable applications and online applications. The applications 114a, 114b, 114c may be for example, but are not limited to, health monitoring applications, blood sugar applications, diet applications, exercise applications, calendar applications, GPS applications, messaging applications, and social media applications, etc. For example, the applications 114a, 114b, 114c may be, but are not limited, Apple® Health, Google Fit®, Facebook®, Instagram®, LinkedIn®, Snapchat®, Gmail®, Lotus Notes®, Microsoft Outlook®, Google® Maps, Waze®, and Apple® Maps, etc. While only a single application 114a, 114b, 114c are illustrated for each of the user devices 110a, 110b, 110c, respectively, it can be appreciated that any number of applications may be part of the glucose management system 100 including one or more than one depending on the user. For example, the user device 110a may be an Apple Watch® which has multiple applications such as, but not limited to, a heartbeat monitor, an exercise tracker, email, and, a calendar, etc. The data associated with the applications 114a, 114b, 114c may be stored on the secondary server 120 associated with the applications 114a, 114b, 114c as application data 126. Alternatively, the data associated with the applications 114a, 114b, 114c, i.e. the application data 126, may be stored on the user databases 116a, 116b, 116c, associated with the user devices 110a, 110b, 110c.

The user databases 116a, 116b, 116c may store any data associated with the user devices 110a, 110b, 110c including, but not limited to, audio, visual, and textual files. Further, the user databases 116a, 116b, 116c may store the data associated with the applications 114a, 114b, 114c, i.e. the application data 126. The user databases 116a, 116b, 116c are described in more detail with reference to FIG. 3.

The secondary server 120 may include an application database 124. While only a single secondary server 120 is illustrated, it can be appreciated that any number of secondary servers 120 may be part of the glucose management system 100 including one or more depending on the user and the number of the applications 114a, 114b, 114c. In the example embodiment, the secondary server 120 may be a desktop computer, a notebook, a laptop computer, a tablet computer, a thin client, or any other electronic device or computing system capable of storing compiling and organizing audio, visual, or textual content and receiving and sending that content to and from other computing devices, such as the user devices 110a, 110b, 110c and the server 130 via the network 140. In some embodiments, the secondary server 120 may include a collection of devices or data sources. The secondary server 120 is described in more detail with reference to FIG. 3.

The application database 124 may be a collection of the application data 126. The application data 126 may be data associated with the applications 114a, 114b, 114c including, but not limited to, audio, visual, and textual files. For example, the application data 126 may include, but is not limited to, blood sugar data, exercise data, health data, email data, social media data, calendar data, and GPS data, etc. associated with the applications 114a, 114b, 114c. The application data 126 stored in application database 124 located on the secondary server 120 or on the user devices 110a, 110b, 110c, or on other secondary databases which may be accessed through the network 140. The application database 124 is described in more detail with reference to FIG. 3.

The server 130 may include the glucose management program 132 and the program database 134. In the example embodiment, the server 130 may be a desktop computer, a notebook, a laptop computer, a tablet computer, a thin client, or any other electronic device or computing system capable of storing compiling and organizing audio, visual, or textual content and receiving and sending that content to and from other computing devices, such as the user devices 110a, 110b, 110c and the secondary server 120 via the network 140. In an example embodiment, the server 130 may be resident in the user devices 110a, 110b, 110c. In yet another embodiment, the server 130 may be separate from the user devices 110a, 110b, 110c and may reside at a secondary location communicating with the user devices 110a, 110b, 110c via the network 140. The server 130 is described in more detail with reference to FIG. 3.

The glucose management program 132 is a program capable of predicting changes in a user's blood glucose levels based on the user's past activity and associated blood glucose levels and recommending actions to be taken by the user to maintain a healthy blood glucose level. The glucose management program 132 may collect the application data 126 from the user databases 116a, 116b, 116c on the user devices 110a, 110b, 110c and/or the application database 124 on the secondary server 120 associated with a user. The glucose management program 132 may identify one or more user activities from the application data 126 and analyze the user's blood glucose data, also contained within the application data 126, to identify the user's typical blood glucose level for an identified activity. Thus, the glucose management program 132 learns in which context and time when a user's blood glucose level is normal, increased, and/or decreased. The glucose management program 132 may also analyze the application data 126 to determine present and future planned user activities. The glucose management program 132 may predict possible changes in the user's blood glucose level for the determined present and future activities based on the user's past activities and associated blood glucose levels. The glucose management program 132 may then recommend a course of action to be taken by the user to maintain a healthy blood glucose level during the determined present and future activities. The glucose management program 132 is described in more detail with reference to FIG. 1b.

The program database 134 may include the user profile 136. The program database 134 may contain any data collected, used, and/or created by the glucose management program 132 including, but not limited to, audio, visual, and textual files. For example, the program database 134 may include, but is not limited to, data collected from the user devices 110a, 110b, 110c and the application data 126. The user profile 136 may include data collected, used, and/or created by the glucose management program 132 including, but not limited to, the lifestyles of a user, user habits, user age, user weight, user diet, user exercise, user geography, user health conditions, user tastes, and user preferences, etc. In another embodiment of the invention, a user may input data into the user profile 136 using the user devices 110a, 110b, 110c via the user interface 112a, 112b, 112c. The program database 134 is described in more detail above and with reference to FIG. 3.

FIG. 1b illustrates example modules of glucose management program 132. In an example embodiment, the glucose management program 132 may include six modules: data collection module 150, user profile module 152, data analysis module 154, user analysis module 156, glucose prediction module 158, and recommendation module 160.

The data collection module 150 collects the applications data 126 associated with a user from the user devices 110a, 110b, 110c and/or the secondary server 120. In one embodiment, the data collection module 150 may store the application data 126 collected from user devices 110a, 110b, 110c and/or the secondary server 120 on the program database 134. Alternatively, the data collection module 150 may read the application data 126 from the user devices 110a, 110b, 110c and/or the secondary server 120.

The user profile module 152 receives data associated with a user from the user devices 110a, 110b, 110c and/or the secondary server 120 to create the user profile 136. The user profile 136 may include data such as, but not limited to, the lifestyles of a user, user habits, user age, user weight, user diet, user exercise, user geography, user health conditions, user tastes, and user preferences, user emergency contact information, etc. A user may enter his/her user profile data using the user interfaces 112a, 112b, 112c on the user devices device 110a, 110b, 110c. The user profile 136 and the data associated with the user profile data 136 may be saved in the program database 134. In another embodiment of the invention, the user profile data associated with the user profile 136 may include data collected, used, and/or created by the glucose management program 132.

The data analysis module 154 analyzes the application data 126 and identifies one or more user activities and the user's blood glucose levels associated with the one or more activities. The data analysis module 154 may determine the user's blood glucose coefficient before and after certain activities, such as, but not limited to, exercising, eating, drinking, etc. For example, the data analysis module 154 may determine that the user's normal blood glucose coefficient is 5.5 mmol/L, which is healthy. The data analysis module 154 may identify how certain activities effect the user's blood glucose coefficient. For example, the user may indicate in a diet tracking application that the user consumed a soda with a high sugar content and a blood glucose tracking device may simultaneously detect an increase in the user's blood glucose level. Thus, the data analysis module 154 may determine that the user's blood glucose coefficient increases when the user drinks a soda with a high sugar content. In another example, the data analysis module 154 may analyze a user's GPS data and social media data and determine that the user is more likely to consume food or drinks with higher sugar contents resulting in increased blood glucose levels when the user is in specific locations or with specific people. For example, the data analysis module 154 may determine that a user is more likely to consume food or drinks with higher sugar contents when the user is visiting his/her parents or travelling away from home. In another example, the data analysis module 154 may analyze a user's social media data and determine that a user is more likely to consume fast food when the user is with a specific person. Thus, the data analysis module 154 learns what types of food and drinks and how certain activities such as, but not limited to, travel and exercise effect the user's blood glucose level. In one embodiment of the invention the data analysis module 154 may utilize existing machine learning systems to analyze the application data 126 and identify one or more user activities and the user's blood glucose levels associated with the one or more activities

The user analysis module 156 analyzes the application data 126 to determine a user's current status. The user's current status may include, but is not limited to, the user's current location, and the user's current blood glucose level. For example, the user analysis module 156 may analyze GPS data contained within the application data 126 from the user device 110a, e.g. a smartphone, and determine the user is currently as a restaurant. Further, user analysis module 156 may analyze blood glucose data contained within the application data 126 from the user device 110b, e.g. a glucometer, and determine that the user's blood glucose level us currently within the normal range. The recommendation module 160 may utilize this data to make a recommendation, which is described in more detail below with reference to the recommendation module 160.

The glucose prediction module 158 analyzes the application data 126 for future planned activities and predicts changes that may occur to a user's blood glucose levels based previous instances of those activities. The glucose prediction module 158 may analyze the application data 126, such as, but not limited to, calendar data, email data, smartphone data, etc. to determine a future activity of a user. The glucose prediction module 158 may determine from the application data 126 that user for example, but not limited to, plans to travel, plans to eat out, plans to exercise, etc. The glucose prediction module 158 may utilize the analyzed data from the data analysis module 154 to predict future changes to a user's blood glucose level in response to a certain planned activity. For example, the data analysis module 154 may determine that a user is more likely to consume food or drinks with higher sugar contents when the user is visiting his/her parents. The glucose prediction module 158 module may determine, from the user's calendar data, that the user is planning to visit his/her parents in a few weeks and thus the user's blood glucose levels are likely to increase at that time. The recommendation module 160 may utilize this data to make a recommendation, which is described in more detail below with reference to the recommendation module 160.

The recommendation module 160 generates a recommendation based on the user profile 136 and the application data 126 analyzed by the glucose management program 132. The recommendation module 160 may generate a recommendation and display that recommendation via the user interface 112a, 112b, 112c on the user device 110a, 110b, 110c. The recommendation may be any type of electronic notification such as, but not limited to, a pop-up notification, an email, a message on the user interface 112a, 112b, 112c, etc. The recommendation module 160 may generate a recommendation of actions a user can take to maintain a healthy blood glucose level, such as, but not limited to, avoiding certain foods and/or beverages, following a particular meal plan, exercising, avoiding particular restaurants, recommending alternative restaurants, etc. If the glucose management program 132 is detecting a user's current status and blood glucose level, the recommendation module 160 may generate a recommendation for the user to follow immediately. For example, if the user analysis module 156 determines that a user is currently at a fast food restaurant based on GPS data, the recommendation module 160 may recommend certain food and/or beverage items to the user which will have a minimal effect on the user's blood glucose level. Alternatively, the recommendation module 160 may recommend to that the user avoid eating at the fast food restaurant because based on past instances of eating at the fast food restaurant the user's blood glucose level increased outside a healthy margin. If the glucose management program 132 is determining a future planned activity, the recommendation module 160 may generate a recommendation for the user to follow in the future to maintain a healthy blood glucose level. For example, if the glucose prediction module 158 determines that the user will be travelling to his/her parents' home next week and the user's glucose level tends to increase in such a context, the recommendation module 160 may recommend an adjustment to the user's diet prior to travelling to his/her parent's house to mitigate a potential increase the user's blood glucose level. Further, the recommendation module 160 may generate a recommendation for foods and/or beverages for the user to avoid, which the user typically consumes at his/her parents' house. Further, the recommendation module 160 may alert a user's emergency contacts if the glucose management program 132 determines that a user's blood glucose levels exceed or fall below a certain level. For example, the user profile 136 may indicate a user's spouse as the user's emergency contact and if the user's blood glucose level exceeds a certain level, i.e. is dangerously high, the recommendation module 160 may send a recommendation to the emergency contact.

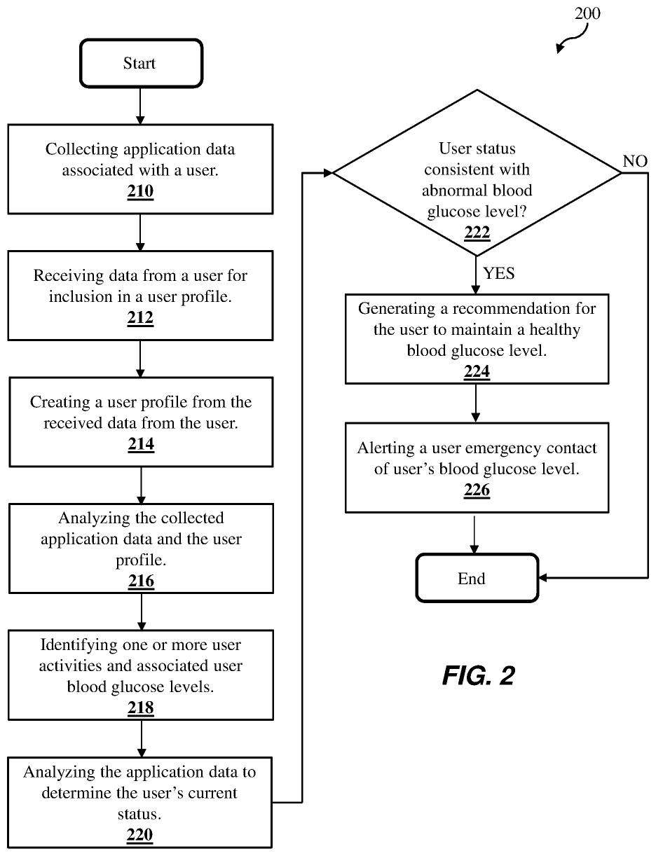

Referring to FIG. 2, a method 200 executed by the glucose management system 100 is depicted, in accordance with an embodiment of the present invention.

Referring to operation 210, the data collection module 150 collects the collects the application data 126 associated with a user from the user devices 110a, 110b, 110c and/or the secondary server 120. Data collection is described in more detail above with reference to the data collection module 150.

Referring to operation 212, the user profile module 152 receives data associated with a user from the user devices 110a, 110b, 110c and/or the secondary server 120. User data receipt is described in more detail above with reference to the user profile module 152.

Referring to operation 214, the user profile module 152 creates the user profile 136 from the received data associated with the user from the user devices 110a, 110b, 110c and/or the secondary server 120. User profile creation is described in more detail above with reference to the user profile module 152.

Referring to operation 216, the data analysis module 154 analyzes the application data 126 for one or more user activities and the user's blood glucose levels associated with the one or more activities. Data analysis is described in more detail above with reference to the data analysis module 154.

Referring to operation 218, the data analysis module 154 identifies one or more user activities and the user's blood glucose levels associated with the one or more activities. Activity identification is described in more detail above with reference to the data analysis module 154.

Referring to operation 220, the user analysis module 156 analyzes the application data 126 to determines the user's current status. Current status determination is described in more detail above with reference to the user analysis module 156.

Referring to operation 222, the glucose management program 132 determines if the current status of the user is consistent with an abnormal user blood glucose level. If the glucose management program 132 determines that the current status of the user is consistent with an abnormal blood glucose level, the recommendation module 160 generates a recommendation to the user to maintain a healthy blood glucose level at operation 224. User recommendation is described in more detail above with reference to the recommendation module 160. If the glucose management program 132 determines that the current status of the user is not consistent with an increased blood glucose level, the glucose management program 132 terminates.

Referring to operation 226, the recommendation module 160 alerts a user's emergency contact of an abnormal user blood glucose level. Alert of an emergency contact is described in more detail above with reference to the recommendation module 160.

Referring to FIG. 3, a method 300 executed by the glucose management system 100 is depicted, in accordance with an embodiment of the present invention. FIG. 3 is substantially similar to FIG. 2; however, instead of the glucose management program 132 determining the current status of the user, the glucose management program 132 determines a future activity of the user.

Operations 310-318 are the same as blocks 210-218, respectively.

Referring to operation 320, the glucose prediction module 158 analyzes the application data 126 to determine a future planned activity. Future activity determination is described in more detail above with reference to the glucose prediction module 158.

Referring to operation 322, the glucose prediction module 158 predicts a user blood glucose level associated with the future planned activity. Future user glucose level prediction is described in more detail above with reference to the re glucose prediction module 158.

Referring to operation 324, the glucose management program 132 determines if the future activity of the user is consistent with an abnormal user blood glucose level. If the glucose management program 132 determines that the future activity of the user is consistent with an abnormal blood glucose level, the recommendation module 160 generates a recommendation to the user to maintain a healthy blood glucose level at operation 326. User recommendation is described in more detail above with reference to the recommendation module 160. If the glucose management program 132 determines that the future activity of the user is not consistent with an increased blood glucose level, the glucose management program 132 terminates.

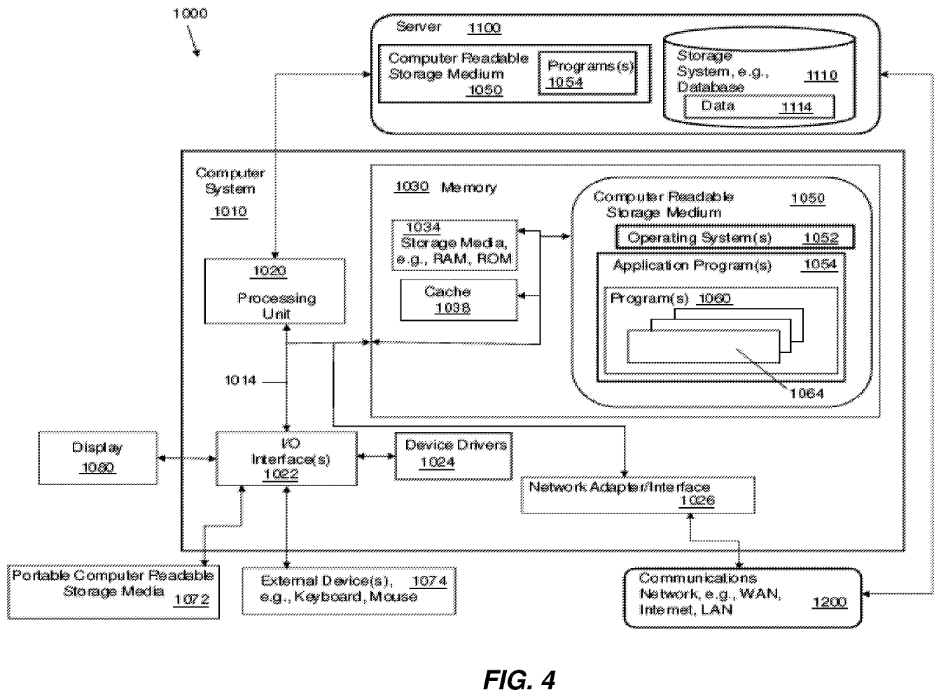

Referring to FIG. 4, a system 1000 includes a computer system or computer 1010 shown in the form of a generic computing device. The methods 200 and 300 for example, may be embodied in a program(s) 1060 (FIGS. 2-3) embodied on a computer readable storage device, for example, generally referred to as memory 1030 and more specifically, computer readable storage medium 1050 as shown in FIG. 4. For example, memory 1030 can include storage media 1034 such as RAM (Random Access Memory) or ROM (Read Only Memory), and cache memory 1038. The program 1060 is executable by the processing unit or processor 1020 of the computer system 1010 (to execute program steps, code, or program code). Additional data storage may also be embodied as a database 1110 which can include data 1114. The computer system 1010 and the program 1060 shown in FIG. 3 are generic representations of a computer and program that may be local to a user, or provided as a remote service (for example, as a cloud based service), and may be provided in further examples, using a website accessible using the communications network 1200 (e.g., interacting with a network, the Internet, or cloud services). It is understood that the computer system 1010 also generically represents herein a computer device or a computer included in a device, such as a laptop or desktop computer, etc., or one or more servers, alone or as part of a datacenter. The computer system can include a network adapter/interface 1026, and an input/output (I/O) interface(s) 1022. The I/O interface 1022 allows for input and output of data with an external device 1074 that may be connected to the computer system. The network adapter/interface 1026 may provide communications between the computer system a network generically shown as the communications network 1200.

The computer 1010 may be described in the general context of computer system-executable instructions, such as program modules, being executed by a computer system. Generally, program modules may include routines, programs, objects, components, logic, data structures, and so on that perform particular tasks or implement particular abstract data types. The method steps and system components and techniques may be embodied in modules of the program 1060 for performing the tasks of each of the steps of the method and system. The modules are generically represented in FIG. 4 as program modules 1064. The program 1060 and program modules 1064 can execute specific steps, routines, sub-routines, instructions or code, of the program.

The method of the present disclosure can be run locally on a device such as a mobile device, or can be run a service, for instance, on the server 1100 which may be remote and can be accessed using the communications network 1200. The program or executable instructions may also be offered as a service by a provider. The computer 1010 may be practiced in a distributed cloud computing environment where tasks are performed by remote processing devices that are linked through a communications network 1200. In a distributed cloud computing environment, program modules may be located in both local and remote computer system storage media including memory storage devices.

More specifically, as shown in FIG. 4, the system 1000 includes the computer system 1010 shown in the form of a general-purpose computing device with illustrative periphery devices. The components of the computer system 1010 may include, but are not limited to, one or more processors or processing units 1020, a system memory 1030, and a bus 1014 that couples various system components including system memory 1030 to processor 1020.

The bus 1014 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The computer 1010 can include a variety of computer readable media. Such media may be any available media that is accessible by the computer 1010 (e.g., computer system, or server), and can include both volatile and non-volatile media, as well as, removable and non-removable media. Computer memory 1030 can include additional computer readable media 1034 in the form of volatile memory, such as random access memory (RAM), and/or cache memory 1038. The computer 1010 may further include other removable/non-removable, volatile/non-volatile computer storage media, in one example, portable computer readable storage media 1072. In one embodiment, the computer readable storage medium 1050 can be provided for reading from and writing to a non-removable, non-volatile magnetic media. The computer readable storage medium 1050 can be embodied, for example, as a hard drive. Additional memory and data storage can be provided, for example, as the storage system 1110 (e.g., a database) for storing data 1114 and communicating with the processing unit 1020. The database can be stored on or be part of a server 1100. Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 1014 by one or more data media interfaces. As will be further depicted and described below, memory 1030 may include at least one program product which can include one or more program modules that are configured to carry out the functions of embodiments of the present invention.

The methods 200, 300 (FIGS. 2-3), for example, may be embodied in one or more computer programs, generically referred to as a program(s) 1060 and can be stored in memory 1030 in the computer readable storage medium 1050. The program 1060 can include program modules 1064. The program modules 1064 can generally carry out functions and/or methodologies of embodiments of the invention as described herein. For example, the program modules 1064 can include the modules 150-160 described above with reference to FIG. 1b. The one or more programs 1060 are stored in memory 1030 and are executable by the processing unit 1020. By way of example, the memory 1030 may store an operating system 1052, one or more application programs 1054, other program modules, and program data on the computer readable storage medium 1050. It is understood that the program 1060, and the operating system 1052 and the application program(s) 1054 stored on the computer readable storage medium 1050 are similarly executable by the processing unit 1020.

The computer 1010 may also communicate with one or more external devices 1074 such as a keyboard, a pointing device, a display 1080, etc.; one or more devices that enable a user to interact with the computer 1010; and/or any devices (e.g., network card, modem, etc.) that enables the computer 1010 to communicate with one or more other computing devices. Such communication can occur via the Input/Output (I/O) interfaces 1022. Still yet, the computer 1010 can communicate with one or more networks 1200 such as a local area network (LAN), a general wide area network (WAN), and/or a public network (e.g., the Internet) via network adapter/interface 1026. As depicted, network adapter 1026 communicates with the other components of the computer 1010 via bus 1014. It should be understood that although not shown, other hardware and/or software components could be used in conjunction with the computer 1010. Examples, include, but are not limited to: microcode, device drivers 1024, redundant processing units, external disk drive arrays, RAID systems, tape drives, and data archival storage systems, etc.

It is understood that a computer or a program running on the computer 1010 may communicate with a server, embodied as the server 1100, via one or more communications networks, embodied as the communications network 1200. The communications network 1200 may include transmission media and network links which include, for example, wireless, wired, or optical fiber, and routers, firewalls, switches, and gateway computers. The communications network may include connections, such as wire, wireless communication links, or fiber optic cables. A communications network may represent a worldwide collection of networks and gateways, such as the Internet, that use various protocols to communicate with one another, such as Lightweight Directory Access Protocol (LDAP), Transport Control Protocol/Internet Protocol (TCP/IP), Hypertext Transport Protocol (HTTP), Wireless Application Protocol (WAP), etc. A network may also include a number of different types of networks, such as, for example, an intranet, a local area network (LAN), or a wide area network (WAN).

In one example, a computer can use a network which may access a website on the Web (World Wide Web) using the Internet. In one embodiment, a computer 1010, including a mobile device, can use a communications system or network 1200 which can include the Internet, or a public switched telephone network (PSTN) for example, a cellular network. The PSTN may include telephone lines, fiber optic cables, microwave transmission links, cellular networks, and communications satellites. The Internet may facilitate numerous searching and texting techniques, for example, using a cell phone or laptop computer to send queries to search engines via text messages (SMS), Multimedia Messaging Service (MMS) (related to SMS), email, or a web browser. The search engine can retrieve search results, that is, links to websites, documents, or other downloadable data that correspond to the query, and similarly, provide the search results to the user via the device as, for example, a web page of search results.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

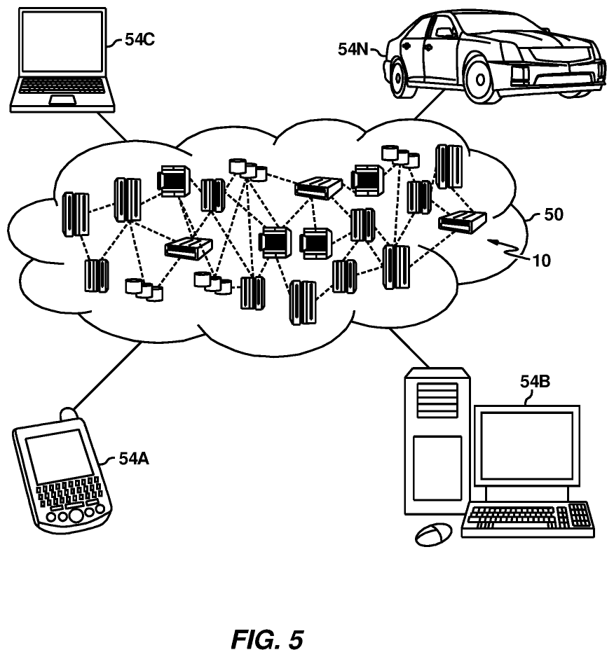

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

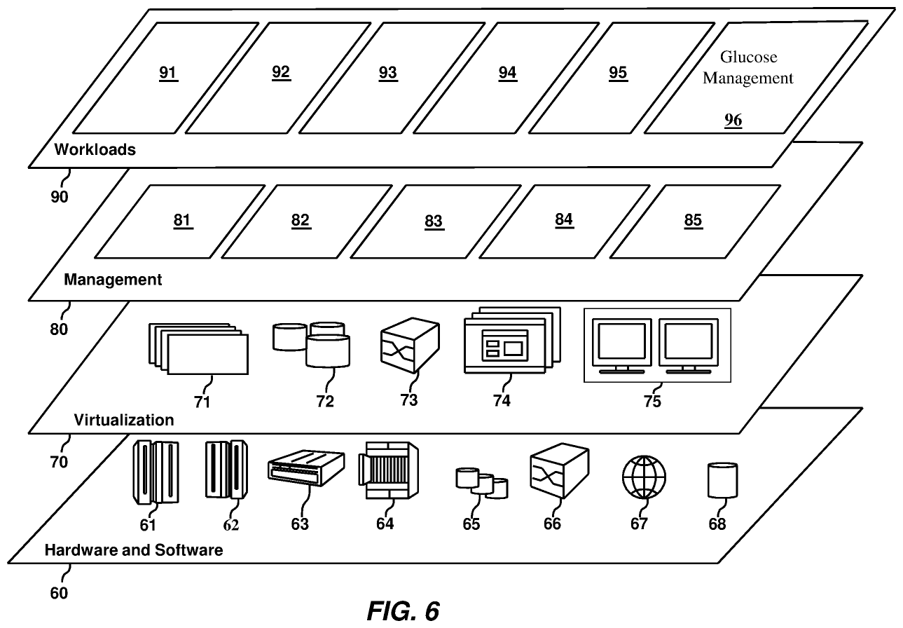

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and glucose management 96.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While steps of the disclosed method and components of the disclosed systems and environments have been sequentially or serially identified using numbers and letters, such numbering or lettering is not an indication that such steps must be performed in the order recited, and is merely provided to facilitate clear referencing of the method's steps. Furthermore, steps of the method may be performed in parallel to perform their described functionality.

What is claimed is:

1. A method for a glucose management, the method comprising:
collecting, by a computer device, application data associated with a user;
receiving, by the computing device, data from the user,
creating, by the computing device, a user profile from the received data from the user;
analyzing, by the computing device, the collected application data and the user profile; and
identifying, by the computing device, one or more user activities and associated blood glucose levels for the user for the one or more activities.

2. A method as in claim 1, further comprising:
analyzing, by the computer device, the application data to determine a current status of the user;
determining, by the computing device, the user's current status is associated with an abnormal blood glucose level; and
generating, by the computing device, in response to determining the user's current status is associated with an abnormal user blood glucose level, a recommendation for maintaining a normal blood glucose level for display to the user.

3. A method as in claim 1, further comprising:
alerting, by the computing device, in response to determining the user's current status is associated with an abnormal user blood glucose level, a user emergency contact of the user's abnormal blood glucose level.

4. A method as in claim 1, further comprising:
analyzing, by the computing device, the application data to determine a future planned activity of the user; and
predicting, by the computing device, a user blood glucose level associated with the future planned activity of the user.

5. A method as in claim 4, further comprising:
determining, by the computing device, the future planned activity is associated with an abnormal blood glucose level; and
generating, by the computing device, in response to determining the future planned activity is associated with an abnormal user blood glucose level, a recommendation for maintaining a normal blood glucose level for display to the user.

6. A method as in claim 1, wherein the application data is collected from one or more user devices, the one or more user devices consisting of one or more of the group consisting of: a blood glucose monitor, a smartwatch, a smartphone, a pedometer, a water tracking device, a global positioning system (GPS), a calorie counter, a beverage sugar monitor, and a weight monitor.

7. A method as in claim 1, wherein analyzing, by the computing device, the collected application data and the user profile, the computer device uses a machine learning engine.

8. A method as in claim 2, wherein the application data includes one or more of the group consisting of: blood glucose data, exercise data, health data, email data, social media data, calendar data, and GPS data, and wherein the user's current status includes one or more of the group consisting of: the user's current location, and the user's current blood glucose level.

9. A computer program product for glucose management, the computer program product comprising:
a computer-readable storage medium having program instructions embodied therewith, wherein the computer readable storage medium is not a transitory signal per se, the program instructions comprising:
program instructions to collect, by a computer device, application data associated with a user;
program instructions to receive, by the computing device, data from the user,
program instructions to create, by the computing device, a user profile from the received data from the user;
program instructions to analyze, by the computing device, the collected application data and the user profile; and
program instructions to identify, by the computing device, one or more user activities and associated blood glucose levels for the user for the one or more activities.

10. A computer program product as in claim 9, further comprising:
program instructions to analyze, by the computer device, the application data to determine a current status of the user;
program instructions to determine, by the computing device, the user's current status is associated with an abnormal blood glucose level; and
program instructions to generate, by the computing device, in response to determining the user's current status is associated with an abnormal user blood glucose level, a recommendation for maintaining a normal blood glucose level for display to the user.

11. A computer program product as in claim 9, further comprising:
program instructions to alert, by the computing device, in response to determining the user's current status is associated with an abnormal user blood glucose level, a user emergency contact of the user's abnormal blood glucose level.

12. A computer program product as in claim 9, further comprising:
program instructions to analyze, by the computing device, the application data to determine a future planned activity of the user; and
program instructions to predict, by the computing device, a user blood glucose level associated with the future planned activity of the user.

13. A computer program product as in claim 12, further comprising:
program instructions to determine, by the computing device, the future planned activity is associated with an abnormal blood glucose level; and
program instructions to generate, by the computing device, in response to determining the future planned activity is associated with an abnormal user blood glucose level, a recommendation for maintaining a normal blood glucose level for display to the user.

14. A computer program product as in claim 9, wherein the application data is collected from one or more user devices, the one or more user devices consisting of one or more of the group consisting of: a blood glucose monitor, a smartwatch, a smartphone, a pedometer, a water tracking device, a global positioning system (GPS), a calorie counter, a beverage sugar monitor, and a weight monitor.

15. A computer program product as in claim 10, wherein the application data includes one or more of the group consisting of: blood glucose data, exercise data, health data, email data, social media data, calendar data, and GPS data, and wherein the user's current status includes one or more of the group consisting of: the user's current location, and the user's current blood glucose level.

16. A computer system for glucose management, the system comprising:
one or more computer processors, one or more computer-readable storage media, and program instructions stored on one or more of the computer-readable storage media for execution by at least one of the one or more processors, the program instructions comprising:
program instructions to collect, by a computer device, application data associated with a user;
program instructions to receive, by the computing device, data from the user, program instructions to create, by the computing device, a user profile from the received data from the user;
program instructions to analyze, by the computing device, the collected application data and the user profile; and
program instructions to identify, by the computing device, one or more user activities and associated blood glucose levels for the user for the one or more activities.

17. A computer system as in claim 16, further comprising:
program instructions to analyze, by the computer device, the application data to determine a current status of the user;
program instructions to determine, by the computing device, the user's current status is associated with an abnormal blood glucose level; and
program instructions to generate, by the computing device, in response to determining the user's current status is associated with an abnormal user blood glucose level, a recommendation for maintaining a normal blood glucose level for display to the user.

18. A computer system as in claim 16, further comprising:
program instructions to alert, by the computing device, in response to determining the user's current status is associated with an abnormal user blood glucose level, a user emergency contact of the user's abnormal blood glucose level.

19. A computer system as in claim 16, further comprising:
program instructions to analyze, by the computing device, the application data to determine a future planned activity of the user; and
program instructions to predict, by the computing device, a user blood glucose level associated with the future planned activity of the user.

20. A computer system as in claim 19, further comprising:
program instructions to determine, by the computing device, the future planned activity is associated with an abnormal blood glucose level; and
program instructions to generate, by the computing device, in response to determining the future planned activity is associated with an abnormal user blood glucose level, a recommendation for maintaining a normal blood glucose level for display to the user.
